(19) United States
(12) Patent Application Publication
(10) Pub. No.: US 2012/0134946 A1
(43) Pub. Date: May 31, 2012

(54) COMPOSITION HAVING ANTIOXIDANT AND WHITENING EFFECTS CONTAINING A CONCENTRATE OF KOREAN RICE WINE

(75) Inventors: Dong Hyun Kim, Suwon-si Gyeonggi-do (KR); Ji Hyun Bae, Seoul (KR); Chinhan Kim, Seoul (KR); Go Un Han, Yongin-si Gyeonggi-do (KR); JiEun Kim, Seoul (KR); So-Woong Choi, Seoul (KR); Chang Man Park, Yongin-si (KR); Duck Hee Kim, Seoul (KR); Han Kon Kim, Suwon-si Gyeonggi-do (KR); Jun Seong Park, Suwon-si Gyeonggi-do (KR)

(73) Assignee: AMOREPACIFIC CORPORATION, Seoul (KR)

(21) Appl. No.: 13/389,305

(22) PCT Filed: Aug. 9, 2010
(86) PCT No.: PCT/KR2010/005207

§ 371 Date: Feb. 7, 2012

(30) Foreign Application Priority Data
Aug. 10, 2009 (KR) 10-2009-0073295

(51) Int. Cl. A61K 8/99; A61P 39/06; A61Q 19/00; A61K 36/899; A61Q 5/08

(52) U.S. Cl. 424/62; 424/195.16

(57) ABSTRACT

Disclosed is a composition containing a concentrate of Korean rice wine as an active ingredient. The composition exhibits antioxidant and whitening effects, and thus can be variously used in the fields of cosmetics, functional food or medicine.

### TECHNICAL FIELD

The present disclosure relates to concentrated makgeolli (Korean rice wine) having antioxidant and whitening effects.

### BACKGROUND ART

Modern people are suffering from various kinds of stress because of rapid change in lifestyles, environmental pollution, or the like. Under stress, generation of reactive oxygen species (ROS) increases in the human body. The reactive oxygen species are known as a cause of facilitating aging. Especially, peroxides including lipid peroxides produced as a result of lipid peroxidation by the reactive oxygen species cause various functional disorders and diseases by inducing oxidative damage to cells.

Several factors are involved in determining human skin color. Among them, activity of melanin-producing melanocytes, distribution of blood vessels, skin thickness, presence or absence of pigments such as carotenoid, bilirubin, etc., and so forth are important. In excess, the melanin pigment may cause live spots, freckles, senile spots, hyperpigmentation, or the like. Also, as the leisure time spent outdoors increases, the need of preventing melanin pigmentation caused by UV is also increasing.

### DISCLOSURE

### Technical Problem

The present disclosure is directed to providing a composition for external application to the skin containing concentrated makgeolli.

The present disclosure is also directed to providing an antioxidant composition containing concentrated makgeolli.

The present disclosure is also directed to providing a whitening composition containing concentrated makgeolli.

### Technical Solution

The compositions according to the present disclosure contain concentrated makgeolli as an active ingredient.

### Advantageous Effects

The compositions according to the present disclosure exhibit antioxidant and whitening effects, and thus can be variously used in the fields of cosmetics, functional food or medicine.

### BEST MODE

The present disclosure relates to a composition comprising concentrated makgeolli (Korean rice wine) as an active ingredient. Through diverse repeated experiments, the inventors of the present disclosure have found out that concentrated makgeolli has antioxidant and whitening effects. The concentrated makgeolli is obtained, for example, by filtering or centrifuging makgeolli and concentrating the resulting filtrate or supernatant.

The makgeolli used in the present disclosure is not specially limited and may be prepared, for example, as follows. A general preparation procedure of makgeolli consists of polishing/washing of rice→soaking/dehydration→steaming and cooling→malting/preparation of crude liquor→first fermentation→second fermentation→maturing→filtration. Details are as follows.

In the step of polishing/washing of rice, rice is polished by a polishing machine and impurities are removed. In the soaking/dehydration step, rice is soaked in water and then water is removed. In the steaming step, the rice containing a suitable amount of water is steamed with hot steam of 100° C. or above. In the step of malting/preparation of crude liquor, the steamed rice is malted by artificially culturing fungi. in the first fermentation step, the malted rice is immersed in water to start fermentation. In the second fermentation step, yeast, water and rice starch are added to the fermented crude liquor and makgeolli is prepared about 5-6 hours later. In the maturing step, the makgeolli prepared in the second fermentation step is matured at a temperature of about 25° C. or lower. In the filtration step, the matured makgeolli is filtered through, for example, a sieve.

The method for concentrating thus prepared makgeolli is not particularly limited. For example, it may be concentrated by filtering and/or centrifuging makgeolli, separating residues from the filtrate and then concentrating the separated filtrate under reduced pressure.

The composition according to the present disclosure may be a composition for external application to the skin comprising concentrated makgeolli as an active ingredient. The composition has antioxidant and/or whitening effect.

In an exemplary embodiment, the composition may be an antioxidant composition. Specifically, the concentrated makgeolli included in the composition exerts antioxidant effect by suppressing generation of reactive oxygen species (ROS). The reactive oxygen species refer to unstable oxygen free radicals with strong reactivity. The reactive oxygen species are generated owing to various physical, chemical and environmental factors, including enzymatic factors, reductive metabolism, chemicals, pollutants and photochemical reactions. They are known to cause aging and various diseases including cancer by non-specifically and irreversibly damaging cellular constituents such as lipids, proteins, sugars and DNAs. In excess, the reactive oxygen species lead to toxicity, i.e. oxidative stress. Accordingly, by suppressing the generation of the reactive oxygen species and removing them, the composition according to the present disclosure exhibits antioxidant effect.

In another exemplary embodiment, the composition may be a whitening composition. Specifically, the concentrated makgeolli included in the composition exerts skin whitening effect by suppressing production of melanin and/or improving pigmentation. The melanin pigment is a phenolic polymer substance as complexes of black pigments and proteins and produced by melanocytes. The melanin pigment protects skin by blocking UV from sunlight and, at the same time, protects proteins and genes in the skin by scavenging free radicals. But, the melanin produced in response to stress-related stimulations inside or outside the skin is stable and remains until it is eliminated via keratinization. When the level of melanin is higher than necessary, it may have cosmetically undesirable effects by inducing hyperpigmentation such as live spots, freckles, etc. Accordingly, by suppressing the production of melanin and improving hyperpigmentation, the composition according to the present disclosure exhibits skin whitening effect.

In an exemplary embodiment, the composition may be a composition for external application to the skin, more specifically a cosmetic composition. The content of the concentrated makgeolli in the composition is not specially limited. For example, it may be included in an amount of 0.001-90 wt %, specifically 0.01-30 wt %, more specifically 0.1-10 wt %, based on the total weight of the composition. The above content range of the concentrated makgeolli was determined through repeated experiments.

The composition for external application to the skin according to the present disclosure may be formulated into, for example, emollient lotion, astringent lotion, nourishing lotion, eye cream, nourishing cream, massage cream, cleansing cream, cleansing foam, cleansing water, powder, essence, pack, etc., but without being limited thereto.

In an exemplary embodiment, the composition according to the present disclosure may be an antioxidant composition and/or whitening composition comprising concentrated makgeolli as an active ingredient. The composition may be, for example, a functional food composition or a pharmaceutical composition, but is not particularly limited thereto.

The functional food composition may be formulated into, for example, powder, granule, tablet, capsule, drink, etc.

The functional food composition may further comprise one or more additive, if necessary. The additive may include, for example, fruit juice (concentrated fruit juice, powdered fruit juice, etc.) of grapefruit, apple, orange, lemon, pineapple, banana, pear, etc.; vitamin (water-soluble and oil-soluble vitamins such as retinyl palmitate, riboflavin, pyridoxine, cyanocobalamin, sodium ascorbate, nicotinic acid amide, calcium pantothenate, folic acid, biotin, cholecalciferol, choline bitartrate, tocopherol, β-carotene, etc.); flavor (lemon flavor, orange flavor, strawberry flavor, grapefruit flavor, vanilla essence, etc.); amino acid, nucleic acid and salts thereof (glutamic acid, sodium glutamate, glycine, alanine, aspartic acid, sodium aspartate, inosinic acid, etc.), plant fiber (polydextrose, pectin, xanthan gum, glucomannan, alginic acid, etc.), mineral (sodium chloride, sodium acetate, magnesium sulfate, potassium chloride, magnesium chloride, magnesium carbonate, calcium chloride, dipotassium phosphate, monosodium phosphate, glycero calcium phosphate, ferrous sodium citrate, ferric ammonium citrate, ferric citrate, manganese sulfate, copper sulfate, sodium iodide, potassium sorbate, zinc, manganese, copper, iodine, cobalt, etc.), or the like.

The pharmaceutical composition may further comprise a pharmaceutical adjuvant such as preservative, stabilizer, wetting agent or emulsifier, salt for osmotic control and/or buffer or other therapeutically useful substance, and may be formulated into various oral or parenteral administration forms according to methods known in the art.

Formulations for oral administration include, for example, tablet, pill, hard and soft capsule, liquid, suspension, emulsion, syrup, granule, etc. These formulations comprise a diluent (e.g. lactose, dextrose, sucrose, mannitol, sorbitol, cellulose and glycine) and a lubricant (e.g. silica, talc, stearic acid and its magnesium or calcium salt, and polyethylene glycol), in addition to the active ingredient. The tablet may further comprise a binder such as magnesium aluminum silicate, starch paste, gelatin, tragacanth, methyl cellulose, sodium carboxymethyl cellulose and polyvinylpyrrolidine. As occasion demands, it may also comprise a pharmaceutical additive such as a disintegrant, an absorbent, a colorant, a flavor, a sweetener, etc., e.g. starch, agar, alginic acid or its sodium salt. The tablet may be prepared by the common mixing, granulation or coating method. Formulations for parenteral administration include formulation for external application to the skin, injection, etc. The formulation for external application to the skin may be an ointment, and the injection may be an isotonic aqueous solution or a suspension.

Determination of the administration dosage of the active ingredient is in the level of those skilled in the art. A daily administration dosage of the composition according to the present disclosure may be composition 1-500 mg/kg, specifically 30-200 mg/kg, for an adult although different depending on various factors including age and physical condition of the subject, presence or absence of complications, or the like. The administration may be made once or twice a day. However, the above-described administration dosage does not limit the scope of the present disclosure by any means.

The concentrated makgeolli may be included in an amount of 10-90 wt %, specifically 20-50 wt %, based on the total weight of the composition, although not being particularly limited thereto. This content was determined in consideration that 10-60% of powder and functional ingredients may be included when preparing tablet or soft capsule and 10-90% of powder and functional ingredients may be included when preparing hard capsule. The present disclosure may provide a functional food composition or a pharmaceutical composition comprising 10-90% of concentrated makgeolli.

### MODE FOR INVENTION

The examples and experiments will now be described. The following examples and experiments are for illustrative purposes only and not intended to limit the scope of the present disclosure.

### Example 1

### Preparation of Concentrated Makgeolli

Commercially available makgeolli (5 kg) was filtered through filter cloth and centrifuged to remove residues from the filtrate. The separated filtrate was concentrated under reduced pressure to obtain concentrated makgeolli (50 g).

### Test Example 1

### Antioxidant Effect of Concentrated Makgeolli (DPPH Test)

Antioxidant effect was measured based on the change in absorbance resulting from reduction of the organic radical 1,1-diphenyl-2-picrylhydrazyl (DPPH) (The antioxidant is oxidized.). The antioxidant effect was determined as the concentration at which absorbance is decreased to 50% of control (IC₅₀).

100 μM DPPH solution (in ethanol, 190 μL) was mixed with the concentrated makgeolli prepared in Example 1 or control (10 μL each). The resulting mixture was kept at 37° C. for 30 minutes and absorbance was measured at 540 nm. Trolox, which is widely used as synthetic antioxidant, was used as the control. The DPPH test result is shown in Table 1.

TABLE 1

| Sample | IC₅₀ (ppm) |
|---|---|
| Concentrated makgeolli | 7.45 |
| Trolox | 8.64 |

As seen from Table 1, the concentrated makgeolli has better antioxidant effect than Trolox.

### Test Example 2

### ROS Production Inhibiting Effect Using Fluorescent Substances

Human keratinocyte HaCaT cell line was used. The cells were seeded on a 96-well black plate for fluorescence measurement, with 2.0×10⁴ cells per well, cultured under the condition of 37° C. and 5% CO₂ for 1 day using Dulbecco's modified Eagle's medium (DMEM, FBS 10%) containing penicillin/streptomycin, and then treated with the test sample. The cells were cultured under the condition of 37°C. and 5% CO₂ for 1 day using serum (FBS)-free DMEM containing penicillin/streptomycin treated with the test sample as described in Table 2.

After culturing for 24 hours, after washing with HEPES-buffered control salt solution (HCSS) to remove the remaining medium and adding 20 μM 2′,7′-dichlorodihydro-fluorescein diacetate (DCFH-DA, Molecular Probes, Inc., 100 μL), the cells were cultured under the condition of 37° C. and 5% CO₂ for 20 minutes and then washed with HCSS. Then, after adding HCSS (100 μL) treated with the samples of different concentrations, the fluorescence intensity of the ROS, oxidized dichlorofluorescein (DCF), was measured using a fluorescence plate reader (Ex=485 nm, Em=530 nm). Then, after immediately and 3 hours after irradiation of UVB (30 mJ/cm²), fluorescence intensity was measured using the fluorescence plate reader (Ex=485 nm, Em=530 nm).

Trolox was used as control. The ROS production inhibiting effect (% of control) of each test sample is shown in Table 2.

TABLE 2

| Concentration (ppm) | Concentrated makgeolli | Trolox |
|---|---|---|
| 50 | 43.2 | 58.5 |
| 25 | 51.7 | 73.3 |
| 10 | 61.5 | 74.6 |
| 1 | 68.2 | 76.9 |

As seen from Table 2, the concentrated makgeolli has better ROS production inhibiting effect than Trolox.

### Test Example 3

### Melanin Production Inhibiting Effect Using Mouse Melanocytes

Mouse melanocytes (Mel-Ab cells) derived from C57BL/6 mouse (Dooley, T. P. et al, Skin Pharmacol., 7, pp. 188-200) were cultured under the condition of 37° C. and 5% CO₂ in DMEM containing 10% FBS, 100 nM 2-O-tetradecanoyphorbol-13-acetate and 1 nM cholera toxin. The cultured Mel-Ab cells were detached with 0.25% trypsin-EDTA and cultured in a 24-well plate, with 10⁵ cells/well. From day 2, hydroquinone or the concentrated makgeolli of Example 1 (10 ppm each) was added for 3 consecutive days as test sample. Hydroquinone was used as positive control. Subsequently, after removing the medium and washing with PBS, the cells were suspended in 1 N sodium hydroxide and absorbance was measured at 400 nm. Based on the measurement result, melanin production inhibiting effect was calculated according to Equation 1. The result is shown in Table 3 (Dooley's method).

Melanin production inhibiting effect (%)=100−(Absorbance of test sample/Absorbance of control×100) Equation 1

TABLE 3

| Test sample | Melanin production inhibiting effect (%) |
|---|---|
| Non-treated | 100.0 |
| Concentrated makgeolli | 38.2 |
| Hydroquinone (positive control) | 41.1 |

As seen from Table 3, the concentrated makgeolli shows comparable melanin production inhibiting effect when compared with hydroquinone.

### Test Example 4

### Whitening Effect for Human Skin

The whitening effect of the concentrated makgeolli of Example 1 for human skin was tested as follows.

12 healthy men were used as test subjects. An opaque tape with a hole of 1.5 cm diameter was attached on the upper arm of the test subject and UVB of about 1.5-2 times the minimal erythemal dose was irradiated to induce tanning.

After the UV radiation, 1% concentrated makgeolli of Example 1 (in 7:3 1,3-butylene glycol:ethanol solvent), 1% hydroquinone or 1% solvent (vehicle, negative control) was applied. Then, change in skin color was observed for 10 weeks. The skin color was measured every week using the colorimeter CR2002 (Minolta, Japan).

The difference in skin color (ΔL*) at the initiation of application and after the completion of test was calculated according to Equation 2. The result is given in Table 4. Whitening effect was determined from the ΔL* of the sample-applied portion and the control portion. A ΔL* value of about 2 can be evaluated as distinct whitening effect and one of about 1.5 can be evaluated as positive whitening effect.

ΔL*=L*after the completion of test−L*at the initiation of application Equation 2

TABLE 4

| Test sample | Change in skin color (ΔL*) |
|---|---|
| Concentrated makgeolli | 1.85 ± 0.25 |
| Hydroquinone (positive control) | 1.90 ± 0.11 |
| Solvent (vehicle, negative control) | 0.50 ± 0.15 |

As seen from Table 4, the concentrated makgeolli shows comparable skin whitening effect when compared with hydroquinone. This means that the substance whitens skin by improving pigmentation caused by UV.

The formulation examples will now be described. The following examples are for illustrative purposes only and not intended to limit the scope of the present disclosure.

### Formulation Example 1

### Preparation of Soft Capsule

Concentrated makgeolli (80 mg), vitamin E (9 mg), vitamin C (9 mg), palm oil (2 mg), hydrogenated vegetable oil (8 mg), beeswax (4 mg) and lecithin (9 mg) were mixed to prepare a soft capsule filling solution according to the commonly employed method. Separately from this, a soft capsule sheet was prepared using gelatin (66 wt %), glycerin (24 wt %) and sorbitol 10 (wt %). 400 mg of the filling solution was filled per capsule to prepare a soft capsule containing the composition according to the present disclosure.

### Formulation Example 2

### Preparation of Tablet

Concentrated makgeolli (80 mg), vitamin E (9 mg), vitamin C (9 mg), galacto-oligosaccharide (200 mg), lactose (60 mg) and maltos e (140 mg) were mixed and granulated using a fluidized-bed dryer. After adding sugar ester (6 mg), the resulting mixture (504 mg) was prepared into tablet according to the commonly employed method.

### Formulation Example 3

### Preparation of Drink

Concentrated makgeolli (80 mg), vitamin E (9 mg), vitamin C (9 mg), glucose (10 g), citric acid (0.6 g) and oligosaccharide syrup (25 g) were mixed. After adding purified water (300 mL), the mixture (200 mL) was filled in a bottle and sterilized at 130° C. for 4-5 seconds.

### Formulation Example 4

### Preparation of Granule

Concentrated makgeolli (80 mg), vitamin E (9 mg), vitamin C (9 mg), anhydrous crystalline glucose (250 mg) and starch (550 mg) were mixed, granulated using a fluidized-bed dryer and filled in a pouch.

### Formulation Example 5

### Preparation of Nourishing Lotion

Nourishing lotion was prepared according to the commonly employed method as described in Table 5.

TABLE 5

| Ingredients | Contents (wt %) |
|---|---|
| Purified water | balance |
| Glycerin | 8.0 |
| Butylene glycol | 4.0 |

### Formulation Example 6

### Preparation of Nourishing Cream

Nourishing cream was prepared according to the commonly employed method as described in Table 6.

TABLE 6

| Ingredients | Contents (wt %) |
|---|---|
| Purified water | balance |
| Glycerin | 3.0 |
| Butylene glycol | 3.0 |

### Formulation Example 7

### Preparation of Massage Cream

Massage cream was prepared according to the commonly employed method as described in Table 7.

TABLE 7

| Ingredients | Contents (wt %) |
|---|---|
| Purified water | balance |
| Glycerin | 8.0 |
| Butylene glycol | 4.0 |

### Formulation Example 8

### Preparation of Pack

Pack was prepared according to the commonly employed method as described in Table 8.

TABLE 8

| Ingredients | Contents (wt %) |
|---|---|
| Purified water | balance |
| Glycerin | 4.0 |
| Polyvinyl alcohol | 15.0 |

### Formulation Example 9

### Preparation of Ointment as Formulation for External Application to the Skin

Ointment was prepared according to the commonly employed method as described in Table 9.

TABLE 9

| Ingredients | Contents (wt %) |
|---|---|
| Purified water | balance |
| Glycerin | 8.0 |
| Butylene glycol | 4.0 |

Those skilled in the art will appreciate that the conceptions and specific embodiments disclosed in the foregoing description may be readily utilized as a basis for modifying or designing other embodiments for carrying out the same purposes of the present disclosure.

The composition according to the present disclosure can be variously used in the fields of cosmetics, functional food or medicine.

1. A composition for external application to the skin comprising concentrated makgeolli.

2. A method for preventing oxidation comprising administering the composition for external application to the skin according to claim 1 to a subject in need thereof.

3. A method for whitening skin comprising administering the composition for external application to the skin according to claim 1 to a subject in need thereof.

4. The method according to claim 2, wherein the concentrated makgeolli included in the composition suppresses generation of reactive oxygen species (ROS).

5. The method according to claim 3, wherein the concentrated makgeolli included in the composition suppresses production of melanin or improves pigmentation.

6. The composition for external application to the skin according to claim 1, wherein the composition is a cosmetic composition.

7. The composition for external application to the skin according to claim 6, wherein the concentrated makgeolli is included in the composition in an amount of 0.001-90 wt % based on the total weight of the composition.

8. The composition for external application to the skin according to claim 7, wherein the concentrated makgeolli is included in the composition in an amount of 0.01-30 wt % based on the total weight of the composition.

9. A method for preventing oxidation of a subject comprising administering to the subject an effective amount of concentrated makgeolli that prevents oxidation.

10. A method for whitening skin of a subject comprising administering to the subject an effective amount of concentrated makgeolli that whitens skin.

11. A functional food composition comprising concentrated makgeolli as an active ingredient.

12. A pharmaceutical composition comprising concentrated makgeolli as an active ingredient.
